SEQUENCE LISTING (8 sequences)

SEQ ID NO: 1 - DNA, 23 bp, Artificial Sequence
  unsure (18): Degenerate amplification primers for QRDR from DNA gyrase genes
ccggatgtgcgcgayggnytnaa

SEQ ID NO: 2 - DNA, 31 bp, Artificial Sequence
  unsure (20)(23)(29): Degenerate amplification primers for QRDR from DNA gyrase genes
ggttatgcggcggaatgttngtngccatncc

SEQ ID NO: 3 - DNA, 30 bp, Artificial Sequence
  unsure (22)(28): Degenerate amplification primers for QRDR from DNA gyrase genes
cgaactgtttctggtggaaggngaywsngc

SEQ ID NO: 4 - DNA, 27 bp, Artificial Sequence
  unsure (16)(25): Degenerate amplification primers for QRDR from DNA gyrase genes
atacagcggcggctgngcdatrtanac

SEQ ID NO: 5 - DNA, 26 bp, Artificial Sequence
  unsure (18)(21): Degenerate amplification primers for QRDR from DNA gyrase genes
cgcgatggcctgaaaccngtncarmg

SEQ ID NO: 6 - DNA, 29 bp, Artificial Sequence
  unsure (18)(24)(27): Degenerate amplification primers for QRDR from DNA gyrase genes
aggcgcgcttcggtatanckcatngcngc

SEQ ID NO: 7 - DNA, 20 bp, Artificial Sequence
  unsure (12)(18): Degenerate amplification primers for QRDR from DNA gyrase genes
cagtttgaaggncaracnaa

SEQ ID NO: 8 - DNA, 27 bp, Artificial Sequence
  unsure (22): Degenerate amplification primers for QRDR from DNA gyrase genes
aatatgcgcgccatcgswrtcngcrtc

(19) United States
(12) Patent Application Publication
(10) Pub. No.: US 2004/0048281 A1
(43) Pub. Date: Mar. 11, 2004

(54) Method for amplifying quinolone-resistance-determining-regions and identifying polymorphic variants thereof

(75) Inventors: Leroy L. Voelker, Royersford, PA (US); Edwina I. Wilding, Paoli, PA (US)

Correspondence Address: GLAXOSMITHKLINE, Corporate Intellectual Property - UW2220, P.O. Box 1539, King of Prussia, PA, 19406-0939, US

(73) Assignee: SmithKline Beecham Corporation

(21) Appl. No.: 10/419,669

(22) Filed: Apr. 21, 2003

Related U.S. Application Data
(63) Continuation of application No. 09/815,498, filed on Mar. 23, 2001, now abandoned.
(60) Provisional application No. 60/192,146, filed on Mar. 24, 2000.

(51) Int. Cl. C12Q 1/68; C12P 19/34

(52) U.S. Cl. 435/6; 435/91.2

(57) ABSTRACT

The present invention provides a novel method for obtaining regions of bacterial polynucleotide sequence associated with quinolone resistance. In particular, methods for detecting Quinolone Resistance-Determining Regions (QRDRs) across a broad phylogenetic range in prokaryotes are disclosed.

### FIELD OF THE INVENTION

The present invention provides methods for identifying regions of bacterial polynucleotide sequence associated with quinolone resistance. In particular, the invention provides degenerate primers that are used to identify Quinolone Resistance-Determining Regions (QRDRs) across a broad phylogenetic range in prokaryotes.

### BACKGROUND OF THE INVENTION

PCR is a well known method for amplifying polynucleotide sequences (Saiki, et al., Nature, 324: 163-166 (1986)). Several reviews of the use of a mixture of oligonucleotides with the same number of bases but varying in sequence “degenerate” to PCR amplify DNA with only a limited portion of amino acid sequence have been published (Compton, et al., PCR Protocols: A Guide to Methods and Applications, Academic Press, 39-45 (1990), Kirchhoff, et al., Methods Mol. Biol., 57: 323-333 (1996)). The most common use of degenerate oligonucleotides is in the amplification of new or uncharacterized nucleotide sequences related to a known family of genes. In the case of bacteria, this approach has been successfully used to amplify unknown sequences from species that are very closely related phylogenetically to those used in the design of the degenerate oligonucleotides. Many investigators have used degenerate oligonucleotides to amplify the Quinolone Resistance-Determining Region (QRDR) from the DNA gyrase and topoisomerase IV genes of bacteria (Okuda, et al., Antimicrobial Agents & Chemotherapy, 43:1156-1162 (1999), Munoz, et al., Antimicrobial Agents Chemotherapy, 40:2252-2257 (1996), Revel, et al., Antimicrobial Agents Chemotherapy, 38:1991-1996 (1994)). However, by contrast to the present invention, these reports have only had success with oligonucleotide pairs that were designed from known sequence of bacterial species closely related to the ones of interest. The present invention solves this problem by using a single “degenerate primer” pair to amplify uncharacterized QRDR sequences from any prokaryote, even very distantly related prokaryotes. This is demonstrated in the Examples as well as being disclosed elsewhere herein.

Approximately 800 bacterial isolates were identified by having a >4 fold increase in their minimum inhibitory concentration (MIC) for gemifloxacin between the initial and post therapy visits in a clinical trial using this antibiotic. A traditional PCR approach would not work for this study due to the large number of isolates, the variety of bacterial species many of which did not have known sequence information for the DNA gyrase and topoisomerase IV genes, and the short time frame in which to complete the analysis. The only alternative, and on which was solved by the present invention, was a PCR approach that used a single set of degenerate primers for each gene but would amplify the QRDRs from the largest variety of bacterial species. Traditional degenerate primers do not generally allow for the amplification of the desired product from more than a few very closely related species.

### SUMMARY OF THE INVENTION

The success in amplifying QRDRs from many bacterial species of unknown sequence is due to the novel design of the primers. This is a result of combining a conserved portion of sequence combined with a degenerate portion to make up each primer. The 3′ degenerate portion of each primer is an important feature which allows for the versatility of the primer with respect to being able to amplify the desired sequence from a large variety of species. This is not possible with a completely conserved or sequence-specific primer, which requires a great degree of homology to the sequence being amplified. The approach of designing degenerate oligonucleotides with a degenerate half and constant half has been used to amplify methyltransferase genes from eukaryotic DNA (Rose, et al., Nucleic Acids Research, 26(7):1628-1635 (1998)), but not for prokaryotic sequences. The primers in the present invention were designed by taking known DNA gyrase and topoisomerase IV sequences for those bacterial species that were most likely to be isolated during a clinical study. A Clustal (Thompson, et al., Nucl. Acids. Res. 22: 4673-4680 (1994)) alignment was performed on these sequences for each gene (gyrA, gyrB, parC, and parE). The sequences were weighted more towards the gram-positive bacterial sequences than the gram-negative ones because it was anticipated that the majority of isolates would be gram-positive bacteria. The skilled artisan can weight such analyses as appropriate in any given situation. The alignments from each gene were then analyzed with the CODEHOP algorithm to identify which regions contained the least amount of degeneracy DNA (Rose, et al., Nucleic Acids Research, 26(7):1628-1635 (1998)).

Once these regions were identified, they were scanned by eye to identify a preliminary region for primer design. These sub-regions were then examined with a computer to rule out any potential cross-hybridization with the other three gene members. Once a region was identified that met all of the criteria, sets of degenerate primers were identified. The skilled artisan can readily select appropriate pairs based on such analyses. After a single degenerate primer had been selected, it was tested against consensus alignment of the input sequences to identify the most common binding site. A non-degenerate string of nucleotide bases was then chosen from the area upstream of the degenerate primer binding site to be added to the 5′ end. The 5′ constant region and the degenerate 3′ half then make up the final primer sequence used.

Cycling conditions were determined using the respective primer sets and DNA samples obtained. Adjustments in conditions were made as needed for individual samples in order to eliminate the amplification of non-specific regions or generate a product at all.

The invention provides a method for amplifying a polynucleotide sequence of a QRDR region comprising the steps of (a) providing a composition comprising a degenerate forward primer of the invention and degenerate reverse primer of the invention, and a sample suspected to have a polynucleotide comprising a QRDR and (b) amplifying a QRDR.

The invention also provides a method of claim 1, wherein a primer is labeled.

A preferred embodiment of the invention is a method wherein a primer is between 10 and 30 nucleotides in length.

A further preferred embodiment of the invention is a method of claim 1 whereby the amplifying step comprises PCR.

A further preferred embodiment of the invention is a method of claim 1, whereby the amplifying step (b) comprises between about 40 to 50 reaction cycles.

The invention also provides a method for identifying a polymorphic polynucleotide sequence of a QRDR comprising the steps of (a) providing a composition comprising a degenerate forward primer of the invention and a degenerate reverse primer of the invention, and a sample suspected to have a polynucleotide comprising a QRDR region (b) amplifying a QRDR to obtain an amplified product (c) sequencing said amplified product to obtain a first polynucleotide sequence and (d) comparing said first polynucleotide sequence with a second polynucleotide sequence of an amplified product made using said degenerate forward primer primer of the invention and said degenerate reverse primer primer of the invention to identify sequence differences between said first polynucleotide sequence and said second polynucleotide sequence.

The invention also provides a method of claim 6, wherein a primer is labeled.

A preferred embodiment of the invention is a method wherein a primer is between 10 and 30 nucleotides in length.

A further preferred embodiment of the invention is a method of claim 1 whereby the amplifying step comprises PCR.

A further preferred embodiment of the invention is a method of claim 1 whereby the amplifying step (b) comprises between about 40 to 50 reaction cycles.

The invention also provides a method of claim 1 or 6 wherein said QRDR is amplified from a member of the genus selected from the group consisting of Psuedomonas, Enterococcus, Staphylococcus, Escherichia, Acinetobacter, Citrobacter, Corynebacterium, Enterobacter, Klebsiella, Morganella, Micrococcus, Proteus, Providenica, Serratia, and Stenotrophomonas.

A further preferred embodiment of the invention is a method of claim 1 or 6 wherein said QRDR is amplified from a member of the species selected from the group consisting of Psuedomonas aeruginosa, Enterococcus faecalis, Staphylococcus aureus, Staphylococcus epidermidis, Escherichia coli, Acinetobacter baumanii, Acinetobacter calcoaceticus, Citrobacter freundii, Corynebacterium xerosis, Enterobacter aerogenes, Enterobacter cloacae, Kleibsiella pneumoniae, Kleibsiella oxytoca, Morganella morganii, Micrococcus luteus, Proteus mirabilis, Providenica spp., Serratia marcessens, and Stenotrophomonas maltophilia.

Further provided is a method of claim 1 or 6 wherein said QRDR is from a gene selected from the group consisting of gyrA, gyrB, parC, and parE.

A preferred embodiment of the invention is a composition comprising the primer of claim 14.

The invention also provides polynucleotide primers useful to amplify QRDR regions.

Compositions comprising such primers are further provided.

A preferred embodiment of the invention is a polynucleotide selected from the group consisting of:

[SEQ ID NO:1]

5′-CCGGATGTGCGCGAYGGNYTNAA-3′;

[SEQ ID NO:2]

5′-GGTTATGCGGCGGAATGTTNGTNGCCATNCC-3′;

[SEQ ID NO:3]

5′-CGAACTGTTTCTGGTGGAAGGNGAYWSNGC-3′;

[SEQ ID NO:4]

5′-ATACAGCGGCGGCTGNGCDATRTANAC-3′;

[SEQ ID NO:5]

5′-CGCGATGGCCTGAAACCNGTNCARMG-3′;

[SEQ ID NO:6]

5′-AGGCGCGCTTCGGTATANCKCATNGCNGC-3′;

[SEQ ID NO:7]

5′-CAGTTTGAAGGNCARACNAA-3′; and

[SEQ ID NO:8]

5′-AATATGCGCGCCATCGSWRTCNGCRTC-3′

Further provided is a method for identifying polynucleotide sequences of a QRDR comprising the steps of (a) providing a composition comprising a degenerate primer of the present invention suitable for use in hybridization, which comprises a solid surface on which is immobilized at pre-defined regions thereon a plurality of defined oligonucleotide/polynucleotide sequences for hybridization and (b) the identification, sequencing and characterization of genes which are implicated in disease, infection, or development and the use of such identified genes and the proteins encoded thereby in diagnosis, prognosis, therapy and drug discovery. The methods and compositions of the present invention may be used with solid state support technology to analyze polynucleotide composition and expression. For example, WO09521944 teaches the methods involved in using a composition suitable for use in hybridization which consists of a solid surface on which is immobilized at pre-defined regions thereon a plurality of defined oligonucleotide/polynucleotide sequences for hybridization.

### BRIEF DESCRIPTION OF THE DRAWINGS

FIG. 1 is an agarose gel electrophoresis of QRDR PCR products generated from total DNA of (1) Psuedomonas aeruginosa 17993 v.1 (2) Enterococcus faecalis 17189 v.1 (3) Staphylococcus aureus 21920 v.1 (4) Enterococcus faecalis 21874 v.1 (5) Enterococcus faecalis 17446 v.1 (6) Staphylococcus epidermidis 2523 v.4 (7) Escherichia coli 2189 v.4 with degenerate oligonucleotides to DNA gyrase genes gyrA (A) and gyrB (B). M, 1-kb ladder marker. The gyrA products are ˜457 bp in size and the gyrB products are ˜349 bp.

FIG. 2 is an agarose gel electrophoresis of QRDR PCR products generated from total DNA of (1) Psuedomonas aeruginosa 17993 v.1 (2) Enterococcus faecalis 17189 v.1 (3) Staphylococcus aureus 21920 v.1 (4) Enterococcus faecalis 21874 v.1 (5) Enterococcus faecalis 17446 v.1 (6) Staphylococcus epidermidis 2523 v.4 (7) Escherichia coli 2189 v.4 with degenerate oligonucleotides to topoisomerase IV genes parC (A) and par E (B). M, 1-kb ladder marker. The parC products are ˜269 bp in size and the parE products are ˜520 bp.

FIG. 3 is an agarose gel electrophoresis of QRDR PCR products generated from total DNA. (A) with degenerate oligonucleotides to DNA gyrase gene gyrA (1) Psuedomonas aeruginosa 5003 v.1 (2) Psuedomonas aeruginosa 5003 v.4 (3) Enterococcus faecalis 6712 v.1 (4) Enterococcus faecalis 6712 v.3 (5) Escherichia coli 17797 v.4 (6) Staphylococcus epidermidis 2523 v.4. (B). with degenerate oligonucleotides to DNA gyrase gene gyrB (1) Psuedomonas aeruginosa 5003 v.1 (2) Psuedomonas aeruginosa 5003 v.4 (3) Staphylococcus epidermidis 2523 v.1 (4) Staphylococcus epidermidis 2523 v.4 (5) Proteus mirabilis 2933 v.1 (6) Proteus mirabilis 2933 v.3. M, 1-kb ladder marker. The gyrA products are ˜457 bp in size and the gyrB products are ˜349 bp.

### DETAILED DESCRIPTION OF THE INVENTION

The present invention meets the unfulfilled needs in the art by providing methods for the identification and use of degenerate primers to identify Quinolone Resistance- Determining Regions (QRDRs) across a broad phylogenetic range in prokaryotes. Employing the methods of this invention permits the resulting identification and isolation of such regions by using. The degenerate primers themselves, and/or products identified, if desired, may be employed in the diagnosis or therapy of the disease or infection with which the genes are associated and in the development of new drugs therefor.

The present invention provides a novel, rapid and simple method for identifying polymorphic variants of Quinolone-Resistance-Determining-Regions (herein “QRDR(s)”) from polynucleotides across a broad phylogenetic range in prokaryotes.

Certain embodiments of this method comprise an amplification reaction using two different degenerate primers complementary to a known sequence region (the “known sequence” or “known sequence fragment”) flanking a QRDR. Primers of the invention made be made using known methods of contracted to be made by a commercial entity. These primers are used to obtain a sequence of a QRDR. To obtain such sequence, isolated polynucleotide, such as a selected sequence fragment of an organism's nucleic acid (e.g., genomic DNA or cDNA) is partially digested with restriction enzyme to linearize it for use as a template for amplification. Circularized template may also be used but linear template is preferred. A first degenerate primer (herein “forward degenerate primer” or “first degenerate primer”) is annealed to flanking QRDR sequences either upstream or downstream of the known sequence fragment and a second degenerate primer (herein “reverse degenerate primer” or “second degenerate primer”) is positioned on the other side of the QRDR, so that the primers flank the QRDR. It is preferred in the methods of the invention that the amplifying steps use PCR (Saiki et al., Nature, 324: 163-166 (1986)).

The amplification or annealing temperature is preferably between about 25° C. and 75° C., more preferably between about 50° C. and 70° C., and most preferably about 55° C. The cycle number is preferably between about 1 and 100 cycles, more preferably between about 5 and 60 cycles, and most preferably about 40 cycles. Amplification reactions in a thermocycler are most preferably carried out for 40-50 cycles, especially at 94° C. for 30 seconds, 55° C. for 30 seconds, and 72° C. for 30 seconds. A preferred final incubation step for additional about 5 to 7 minutes, more preferably about 5 minutes, at 72° C. is also provided by the present invention. Degenerate primers designed to amplify the gryA, gyrB, parC and parE genes across a broad phylogenetic range in prokaryotes are provided.

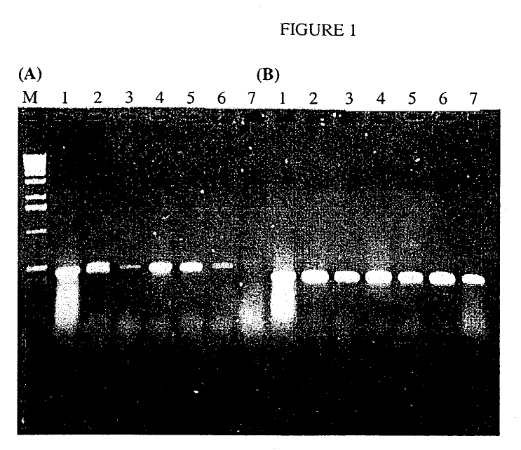

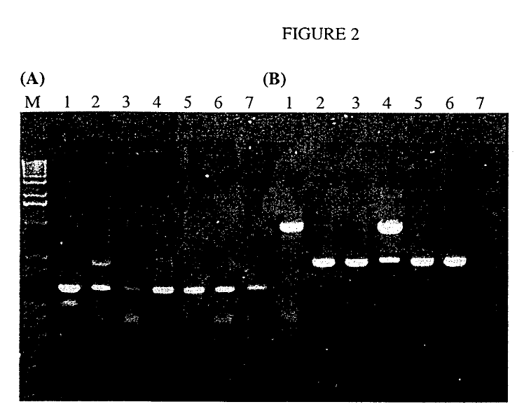

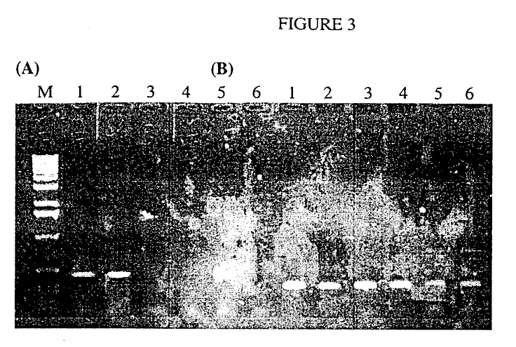

The amplification reaction is, in any given reaction, predicted to generate certain major products, as exemplified in FIGS. 1, 2, and 3. After separation of the amplification products using known methods, such as, for example, by gel electrophoresis and exposure to film, a limited number of labeled bands are identifiable, such as through visual or automated inspection of an autoradiogram (see FIGS. 1 and 2). These fragments can subsequently be cloned into a suitable vector, e.g., pUC19 or pBR322, and sequenced, such as using well-known sequencing methods.

The invention may be used to amplify and determine QRDR sequences across a broad phylogenetic range of prokaryotes. Reverse transcription may be used prior to performing the amplification reactions so that reverse transcriptase copies of RNAs encoded by QRDR sequences may be amplified and the QRDR sequences obtained.

A preferred embodiment of the present invention is directed to amplifying certain QRDR regions of gyrA, gyrB, parC and parE genes from polynucleotides, particularly genomic DNA isolated from bacterial isolates, that were identified by having a ≧4 fold increase in their minimum inhibitory concentration (MIC) for gemifloxacin between the initial and post therapy visits in a clinical trial using this antibiotic. An example of such preferred embodiments are provided as Example 1, 2, and 3. Bacterial polynucleotides, for example DNA, may be isolated from any source, such from a clinical sample or as individual colonies grown on, but not limited to, agar plates. Clinical sample useful in the methods of the invention include, but are not limited to, any material derived from an individual or from an organism infecting, infesting or inhabiting an, individual, including but not limited to, cells, tissues and waste, such as, bone, blood, serum, cerebrospinal fluid, semen, saliva, muscle, cartilage, organ tissue, skin, urine, stool or autopsy materials.

It is preferred that a single colony is resuspended in a volume of buffer, preferably between 10 and 500 μl., more preferably between 10 and 100 μl., and most preferably about 50 μl. The skilled artisan will readily be able to choose a buffer useful for a particular amplification reaction. A preferred buffer is, for example, 50 mM Tris-HCl, pH8.0, 1 mM EDTA buffer. Cell samples may be incubated in a boiling water bath for 10 minutes, then chilled briefly on ice, for between 10 seconds and one or more minutes. Samples thus treated may further be centrifuged, such as at 12,000× g, to pellet cell debris. Preferably, a small volume of supernatant may be used for an amplification reaction, such as a PCR reaction. It is preferred that this volume of supernatant be between 1 μl and 100 μl. It is more preferred that this volume of supernatant be between 1 μl and 10 μl. It is most preferred that this volume of supernatant be 10 μl.

Preferred QRDRs of DNA gyrase genes of the invention, include, but are note limited to, those regions of gyrA and gyrB, and topoisomerase IV genes, parC and parE. Such QRDRs may be amplified by PCR from polynucleotide isolated from an organism and source of choice, such as genomic DNA.

Preferred degenerate primers provided in the invention, include, but are not limited to the following:

| | | |
|---|---|---|
| gyrA Deg.For | 5′-CCGGATGTGCGCGAYGGNYTNAA-3′; | [SEQ ID NO:1] |
| gyrA Deg.Rev | 5′-GGTTATGCGGCGGAATGTTNGTNGCCATNCC-3′′ | [SEQ ID NO:2] |
| gyrB Deg.For | 5′-CGAACTGTTTCTGGTGGAAGGNGAYWSNGC-3′; | [SEQ ID NO:3] |
| gyrB Deg.Rev | 5′-ATACAGCGGCGGCTGNGCDATRTANAC-3′; | [SEQ ID NO:4] |
| parC Deg.For | 5′-CGCGATGGCCTGAAACCNGTNCARMG-3′; | [SEQ ID NO:5] |
| parC Deg.Rev | 5′-AGGCGCGCTTCGGTATANCKCATNGCNGC-3′; | SEQ ID NO:6] |
| parE Deg.For | 5′-CAGTTTGAAGGNCARACNAA-3′; | [SEQ ID NO:7] |
| parE Deg.Rev | 5′-AATATGCGCGCCATCGSWRTCNGCRTC-3′ | [SEQ ID NO:8] |

These primers, and variants thereof, are useful in the methods of the invention, among other methods.

In a further aspect, the present invention provides for an isolated polynucleotide comprising or consisting of a polynucleotide sequence that has at least 95% identity, even more preferably at least 97-99% or exact identity to a polynucleotide of the invention over the entire length of such polynucleotide of the invention, or the entire length of that portion of a polynucleotide of the invention which encodes a region of a QRDR polypeptide, or a variant thereof.

As used herein, “primer(s)” refers to relatively short polynucleotides or oligonucleotides, preferably sequences of about 5 to 50 nucleotides in length. Primers. such as single-stranded DNA oligonucleotides, often are synthesized by chemical methods, such as those implemented on automated oligonucleotide synthesizers. However, primers can be made by a variety of other methods, including in vitro recombinant DNA-mediated techniques and by expression of DNAs in cells and organisms. Initially, chemically synthesized DNAs typically are obtained without a 5′ phosphate. The 5′ ends of such oligonucleotides are not substrates for phosphodiester bond formation by ligation reactions that employ DNA ligases typically used to form recombinant DNA molecules. The 3′ end of a chemically synthesized primer generally has a free hydroxyl group and, in the presence of a ligase, such as T4 DNA ligase, readily will form a phosphodiester bond with a 5′ phosphate of another polynucleotide, such as another oligonucleotide. As is well known, this reaction can be prevented selectively, where desired, by removing the 5′ phosphates of the other polynucleotide(s) prior to ligation.

As used herein, “degenerate primers” refers to primers designed from combining a conserved portion of sequence combined with a degenerate portion to make up each primer. The 3′ degenerate portion of each primer is an important feature which allows for the versatility of the primer with respect to being able to amplify the desired sequence from a large variety of species. This is not possible with a completely conserved or sequence specific primer which requires a great degree of homology to the sequence being amplified.

A completely degenerate polynucleotide would not be feasible for several reasons. Primers designed with enough degeneracy to allow for annealing to a large diversity of target sequences will give too many amplification products in the early rounds of cycling. This results in a depletion of the components in the PCR reaction and does not allow enough of any one product to be made in sufficient quantity. A primer with very little degeneracy would eliminate some of this drawback but because of its low degeneracy will not be capable of binding to a very diverse array of sequences.

The solution to the problem is to design a primer which combines a highly degenerate 3′ end (for greater diversity in sequence binding) with a non-degenerate 5′ end (for greater specificity during amplification). This allows for the primer to bind to an unknown sequence with a lower degree of homology initially followed by amplification of only a small number of products produced during the early cycles of the PCR. The level of homology between the target sequence and the primers 3′ end is what determines how well a given reaction works and whether or not any non-specific products are produced in addition to the desired product.

“Plasmid(s)” generally are designated herein by a lower case p preceded and/or followed by capital letters and/or numbers, in accordance with standard naming conventions that are familiar to those of skill in the art. Starting plasmids disclosed herein are either commercially available, publicly available on an unrestricted basis, or can be constructed from available plasmids by routine application of well known, published procedures. Many plasmids and other cloning and expression vectors that can be used in accordance with the present invention are well known and readily available to those of skill in the art. Moreover, those of skill readily may construct any number of other plasmids suitable for use in the invention. The properties, construction and use of such plasmids, as well as other vectors, in the present invention will be readily apparent to those of skill from the present disclosure.

“Identity,” as known in the art, is a relationship between two or more polypeptide sequences or two or more polynucleotide sequences, as the case may be, as determined by comparing the sequences. In the art, “identity” also means the degree of sequence relatedness between polypeptide or polynucleotide sequences, as the case may be, as determined by the match between strings of such sequences. “Identity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

Parameters for polypeptide sequence comparison include the following: Algorithm:

Needleman and Wunsch, J. Mol Biol. 48: 443-453 (1970)

Comparison matrix: BLOSSUM62 from Hentikoff and Hentikoff, Proc. Natl. Acad. Sci.

USA. 89:10915-10919 (1992)

Gap Penalty: 12

Gap Length Penalty: 4

A program useful with these parameters is publicly available as the “gap” program from Genetics Computer Group, Madison Wis. The aforementioned parameters are the default parameters for peptide comparisons (along with no penalty for end gaps).

Parameters for polynucleotide comparison include the following: Algorithm:

Needleman and Wunsch, J. Mol Biol. 48: 443-453 (1970)

Comparison matrix: matches =+10, mismatch =0

Gap Penalty: 50

Gap Length Penalty: 3

Available as: The “gap” program from Genetics Computer Group, Madison Wis. These are the default parameters for nucleic acid comparisons.

A preferred meaning for “identity” for polynucleotides is provided in (1) below.

(1) Polynucleotide embodiments further include an isolated polynucleotide comprising a polynucleotide sequence having at least a 95, 97 or 100% identity to the reference sequence of SEQ ID NOs:1-8, wherein said polynucleotide sequence may be identical to the reference sequence of SEQ ID NOs:1-8 or may include up to a certain integer number of nucleotide alterations as compared to the reference sequence, wherein said alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence, and wherein said number of nucleotide alterations is determined by multiplying the total number of nucleotides in SEQ ID NOs:1-8 by the integer defining the percent identity divided by 100 and then subtracting that product from said total number of nucleotides in SEQ ID NOs:1-8, or:

nₙ≦xₙ−(xₙ·y),

wherein nₙ is the number of nucleotide alterations, xₙ is the total number of nucleotides in SEQ ID NOs:1-8, y is 0.95 for 95%, 0.97 for 97% or 1.00 for 100%, and · is the symbol for the multiplication operator, and wherein any non-integer product of xₙ and y is rounded down to the nearest integer prior to subtracting it from xₙ.

“Individual(s)” means a multicellular eukaryote, including, but not limited to a metazoan, a mammal, an ovid, a bovid, a simian, a primate, and a human.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Moreover, a polynucleotide or polypeptide that is introduced into an organism by transformation, genetic manipulation or by any other recombinant method is “isolated” even if it is still present in said organism, which organism may be living or non-living.

“Bacteria(ium)” means a prokaryote, including but not limited to, a member of the genus Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillius, Streptobacillus, Listeria, Calymmatobacterium, Brucella, Bacillus, Clostridium, Treponema, Escherichia, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia and Mycoplasma, and further including, but not limited to, a member of the species or group, Group A Streptococcus, Group B Streptococcus, Group C Streptococcus, Group D Streptococcus, Group G Streptococcus, Streptococcus pneumoniae, Streptococcus pyogenes, Streptococcus agalactiae, Streptococcus faecalis, Streptococcus faecium, Streptococcus durans, Neisseria gonorrheae, Neisseria meningitidis, Staphylococcus aureus, Staphylococcus epidermidis, Enterococcus faecalis, Acinetobacter baumanii, Acinetobacter calcoaceticus, Corynebacterium xerosis, Enterobacter aerogenes, Enterobacter cloacae, Kleibsiella oxytoca, Morganella morganii, Micrococcus luteus, Providenica spp., Stenotrophomonas maltophilia, Corynebacterium diptheriae, Gardnerella vaginalis, Mycobacterium tuberculosis, Mycobacterium bovis, Mycobacterium ulcerans, Mycobacterium leprae, Actinomycetes israelii, Listeria monocytogenes, Bordetella pertusis, Bordetella parapertusis, Bordetella bronchiseptica, Escherichia coli, Shigella dysenteriae, Haemophilus influenzae, Haemophilus aegyptius, Haemophilus parainfluenzae, Haemophilus ducreyi, Bordetella, Salmonella typhi, Citrobacter freundii, Proteus mirabilis, Proteus vulgaris, Yersinia pestis, Kleibsiella pneumoniae, Serratia marcessens, Serratia liquefaciens, Vibrio cholera, Shigella dysenterii, Shigella flexneri, Pseudomonas aeruginosa, Francisella tularensis, Brucella abortis, Bacillus anthracis, Bacillus cereus, Clostridium perfringens, Clostridium tetani, Clostridium botulinum, Treponema pallidum, Rickettsia rickettsii and Chlamydia trachomitis.

“Polynucleotide(s)” generally refers to any polyribonucleotide or polydeoxyribonucleotide, that may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that comprise one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

“Variant(s)” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusion proteins and truncations in the polypeptide encoded by the reference sequence, as discussed below.

### EXAMPLES

The present invention is further described by the following examples. The examples are provided solely to illustrate the invention by reference to specific embodiments. These exemplifications, while illustrating certain specific aspects of the invention, do not portray the limitations or circumscribe the scope of the disclosed invention.

All examples were carried out using standard techniques, which are well known and routine to those of skill in the art, except where otherwise described in detail. Routine molecular biology techniques of the following examples can be carried out as described in standard laboratory manuals, such as Sambrook et al., MOLECULAR CLONING: A LABORATORY MANUAL, 2nd Ed.; Cold Spring Harbor Laboratory Press, Cold Spring Harbor, N.Y. (1989).

All parts or amounts set out in the following examples are by weight, unless otherwise specified.

Unless otherwise stated size separation of fragments in the examples below was carried out using standard techniques of agarose and polyacrylamide gel electrophoresis (“PAGE”) in Sambrook et al., MOLECULAR CLONING: A LABORATORY MANUAL, 2nd Ed., Cold Spring Harbor Laboratory Press, Cold Spring Harbor, N.Y. (1989) and numerous other references such as, for instance, by Goeddel et al., Nucleic Acids Res. 8: 4057 (1980).

### Example 1

Amplification of Quinolone-Resistance-Determining-Regions with Degenerate Primers Using an Annealing Temperature of 60° C., 40 Reaction Cycles, and Platinum Taq DNA Polymerase with a “Hot Start”.

An embodiment of the present invention is directed to identifying whether or not clinical isolates have developed resistance to anti-microbial therapies. The clinical isolates were identified by having ≧4 fold increase in their minimum inhibitory concentration (MIC) for gemifloxacin between the initial and post therapy visits in a clinical trial using this antibiotic. This example is directed to amplifying the QRDRs of DNA gyrase genes: gyrA and gyrB; and topoisomerase IV genes: parC and parE from prokaryotic DNA isolated from such clinical isolates.

To demonstrate this embodiment, 2 ml of growth medium (Mueller Hinton Broth, or Todd-Hewitt Broth with 5% yeast extract) was inoculated with a small amount of frozen stock from clinical isolates and incubated at 37° C. overnight. Isolated DNA was obtained from the overnight culture by harvesting the cells at 12,000× g for 5 minutes and washed with 1.0 ml of 50 mM Tris-HCl (pH8.0), 1 mM EDTA buffer. The cells were resuspended in 50 μl of 50 mM Tris-HCl (pH8.0), 1 mM EDTA buffer. Then, 50 μl of lysis buffer (0.45% Tween 20, 0.45% Triton X-100, and 24 ug Proteinase K) was added, and incubated at 56° C. for 1 hour. The sample was boiled for 10 minutes, briefly chilled on ice for one minute, then centrifuged at 12,000× g for 10 minutes to pellet the cell debris. The DNA was ethanol precipitated from the supernatant and resuspended in 100 μl of 50 mM Tris- HCl (pH8.0), 1 mM EDTA buffer.

Five μl of DNA was amplified in a 50 μl standard PCR reaction using the CODEHOP algorithm 60 program (9 min. 94° C. “Hot Start” then 40 cycles of 94° C. (30 sec.), 60° C. (30 sec.), 72° C. (30 sec.), and a final extension at 72° C. for 5 minutes. 5 μl of each reaction was analyzed on a 1% agarose gel.

The Quinolone-Resistance-Determining-Regions (QRDRs) of the DNA gyrase genes, gyrA and gyrB, and topoisomerase IV genes, parC and parE, were amplified by PCR from prokaryotic DNA by using the following mixture.

| | | Final |
|---|---|---|
| Components | Volume (μl) | Concentration |
| Genomic DNA | 5 | — |
| 10× PCR Buffer | 5 | 1× |
| 10 mM dNTP mixture | 1 | 0.2 mM each |
| Primers (25 pmol/μl) | 2 | 100 pmoles each |
| 50 mM MgCl₂ | 1.5 | 1.5 mM |
| Platinum Taq DNA polymerase | 0.5 | 2.5 units |
| Sterile ddH₂O | 35 | — |

A preferred embodiment of the invention uses Platinum Taq DNA Polymerase (Licensed to Life Technologies, Inc. under U.S. Pat. No. 5,338,671) for an automatic “Hot Start” amplification of DNA fragments (Westfall, et al., Focus® 19, 46 (1997)). During the initial denaturation step of PCR, the inhibitor is denatured and active Taq DNA polymerase is released into the reaction (Westfall, et al., Focus 20, 17 (1998)). Platinum Taq DNA Polymerase is recombinant Taq DNA polymerase complexed with proprietary antibody that inhibits polymerase activity. Due to specific binding of the inhibitor, PLATINUM Taq DNA Polymerase is provided in an inactive form. This results in a DNA polymerase which is activated in a temperature dependent manner (at 94° C.) during the start of PCR. This technology reduces the number of initial mispriming events. While using degenerate primers, initial mispriming events occur with a higher frequency than sequence specific primers. This allows one skilled in the art to focus on optimizing the annealing temperatures for degenerate primers that may not work in the first effort since mispriming can still occur during the PCR. The annealing/amplification temperature is preferably between about 25° C. and 75° C., more preferably between about 50° C. and 70° C., and most preferably about 55° C.

Degenerate QRDR primers designed to amplify the gryA, gyrB, parC and parE genes from various bacterial species are shown below.

| | | |
|---|---|---|
| gyrA Deg.For | 5′-CCGGATGTGCGCGAYGGNYTNAA-3′; | [SEQ ID NO:1] |
| gyrA Deg.Rev | 5′-GGTTATGCGGCGGAATGTTNGTNGCCATNCC-3′; | [SEQ ID NO:2] |
| gyrB Deg.For | 5′-CGAACTGTTTCTGGTGGAAGGNGAYWSNGC-3′; | [SEQ ID NO:3] |
| gyrB Deg.Rev | 5′-ATACAGCGGCGGCTGNGCDATRTANAC-3′; | [SEQ ID NO:4] |
| parC Deg.For | 5′-CGCGATGGCCTGAAACCNGTNCARMG-3′; | [SEQ ID NO:5] |
| parC Deg.Rev | 5′-AGGCGCGCTTCGGTATANCKCATNGCNGC-3′; | [SEQ ID NO:6] |
| parE Deg.For | 5′-CAGTTTGAAGGNCARACNAA-3′; | [SEQ ID NO:7] |
| parE Deg.Rev | 5′-AATATGCGCGCCATCGSWRTCNGCRTC-3′ | [SEQ ID NO:8] |

Collectively, FIGS. 1, 2, and 3 illustrate the results of Example 1. The QRDRs were amplified in gyrA, gyrB, parC, and parE genes with an annealing temperature of 60° C., 40 reaction cycles, and Platinum Taq DNA polymerase with a “Hot Start”.

gyrA: Psuedomonas, Enterococcus, Staphylococcus, Escherichia

gyrB: Psuedomonas, Enterococcus, Staphylococcus, Escherichia

parC: Psuedomonas, Enterococcus, Staphylococcus, Escherichia

parE: Enterococcus, Staphylococcus, Escherichia (faint band)

### Example 2

Amplification of Quinolone-Resistance-Determining-Regions with Degenerate Primers Using an Annealing Temperature of 52° C., 40 Reaction Cycles, and Taq DNA Polymerase without a “Hot Start”.

An embodiment of the present invention is directed to identifying whether or not clinical isolates have developed resistance to anti-microbial therapies. The clinical isolates were identified by having a ≧4 fold increase in their minimum inhibitory concentration (MIC) for gemifloxacin between the initial and post therapy visits in a clinical trial using this antibiotic. This example is directed to amplifying the QRDRs of DNA gyrase genes: gyrA and gyrB from prokaryotic DNA isolated from such clinical isolates.

To demonstrate this embodiment, bacterial DNA was isolated from either individual colonies on agar plates inoculated directly from a glycerol stock or 2 ml of growth medium was inoculated with a small amount of frozen stock from clinical isolates and incubated at 37° C. overnight.

DNA was isolated from individual colonies on an agar plate (Trypticase Soy Agar with 5% sheep blood) by resuspending, a single colony in 50 μl of 50 mM Tris-HCl (pH8.0), 1 mM EDTA buffer. The sample was boiled for 10 minutes, briefly chilled on ice for one minute, then centrifuged at 12,000× g for 10 minutes to pellet the cell debris. As a template, 10 μl of the supernatant was used directly for the PCR reaction. Alternatively, the DNA was ethanol precipitated from the supernatant and resuspended in 100 μl of 50 mM Tris-HCl (pH8.0), 1 mM EDTA buffer. If the DNA is precipitated, 5 μl of DNA was used directly for the PCR reaction supplemented with an additional 5 μl of sterile ddH₂O.

DNA was also isolated from overnight cultures (Mueller Hinton Broth, Todd-Hewitt Broth with 5% yeast extract, or Trypticase Soy Broth) by harvesting the cells at 12,000× g for 5 minutes and then washed the pellet with 1.0 ml of 50 mM Tris-HCl (pH8.0), 1 mM EDTA buffer. The cells were resuspended in 50 μl of 50 mM Tris-HCl (pH8.0), 1 mM EDTA buffer. Then, 50 μl of lysis buffer (0.45% Tween 20, 0.45% Triton X-100, and 24 ug Proteinase K) was added, and incubated at 56° C. for 1 hour. The sample was boiled for 10 minutes, briefly chilled on ice for one minute, then centrifuged at 12,000× g for 10 minutes to pellet the cell debris. The DNA was ethanol precipitated from the supernatant and resuspended in 100 μl of 50 mM Tris-HCl (pH8.0), 1 mM EDTA buffer. Five μI of DNA was used directly for the PCR reaction supplemented with an additional 5 μl of sterile ddH₂O.

10 μl of each template was amplified in a 50 μl standard PCR reaction using the CODEHOP algorithm 60 program (9 min. 94° C. (No “Hot Start” (standard Taq)) then 40 cycles of 94° C. (30 sec.), 52° C. (30 sec.), 72° C. (30 sec.), and a final extension at 72° C. for 5 minutes. 5 μl of each reaction was analyzed on a 1% agarose gel.

The Quinolone-Resistance-Determining-Regions (QRDRs) of the DNA gyrase genes, gyrA and gyrB were amplified by PCR from prokaryotic DNA by using the following mixture.

| | | Final |
|---|---|---|
| Components | Volume (μl) | Concentration |
| Genomic DNA | 10 | — |
| 10× PCR Buffer | 5 | 1× |
| 10 mM dNTP mixture | 1 | 0.2 mM each |
| Primers (25 pmol/μl) | 2 | 100 pmoles each |
| 50 mM MgCl₂ | 1.5 | 1.5 mM |
| Taq DNA polymerase (Roche) | 0.5 | 2.5 units |
| Sterile ddH₂O | 30 | — |

Degenerate QRDR primers designed to amplify the gryA and gyrB genes from various bacterial species are shown below.

| | | |
|---|---|---|
| gyrA Deg.For | 5′-CCGGATGTGCGCGAYGGNYTNAA-3′; | [SEQ ID NO:1] |
| gyrA Deg.Rev | 5′-GGTTATGCGGCGGAATGTTNGTNGCCATNCC-3′; | [SEQ ID NO:2] |
| gyrB Deg.For | 5′-CGAACTGTTTCTGGTGGAAGGNGAYWSNGC-3′; | [SEQ ID NO:3] |
| gyrB Deg.Rev | 5′-ATACAGCGGCGGCTGNGCDATRTANAC-3′ | [SEQ ID NO:4] |

Table 1 illustrates which QRDR regions were amplified by the conditions described in Example 2. The following QRDR regions of the DNA gyrase genes gyrA and gyrB where amplified by PCR with an annealing temperature of 52° C., 40 reaction cycles, and Taq DNA polymerase without a “Hot Start”.

### Example 3

Amplification of Quinolone-Resistance-Determining-Regions with Degenerate Primers Using an Annealing Temperature of 55° C., 50 Reaction Cycles, and Taq DNA Polymerase without a “Hot Start”.

An embodiment of the present invention is directed to identifying whether or not clinical isolates have developed resistance to anti-microbial therapies. The clinical isolates were identified by having a ≧4 fold increase in their minimum inhibitory concentration (MIC) for gemifloxacin between the initial and post therapy visits in a clinical trial using this antibiotic. This example is directed to amplifying the QRDRs of the topoisomerase IV gene parC from prokaryotic DNA isolated from such clinical isolates.

To demonstrate this embodiment, bacterial DNA was isolated from either individual colonies on agar plates inoculated directly from a glycerol stock or 2 ml of growth medium was inoculated with a small amount of frozen stock from clinical isolates and incubated at 37° C. overnight.

DNA was isolated from individual colonies on an agar plate (Trypticase Soy Agar with 5% sheep blood) by resuspending, a single colony in 50 μl of 50 mM Tris-HCl (pH8.0), 1 mM EDTA buffer. The sample was boiled for 10 minutes, briefly chilled on ice for one minute, then centrifuged at 12,000× g for 10 minutes to pellet the cell debris. As a template, 10 μl of the supernatant was used directly for the PCR reaction. Alternatively, the DNA was ethanol precipitated from the supernatant and resuspended in 100 μl of 50 mM Tris-HCl (pH8.0), 1 mM EDTA buffer. If the DNA is precipitated, 5 μl of DNA was used directly for the PCR reaction supplemented with an additional 5 μl of sterile ddH₂O.

DNA was also isolated from overnight cultures (Mueller Hinton Broth, Todd-Hewitt Broth with 5% yeast extract, or Trypticase Soy Broth) by harvesting the cells at 12,000× g for 5 minutes and then washed the pellet with 1.0 ml of 50 mM Tris-HCl (pH8.0), 1 mM EDTA buffer. The cells were resuspended in 50 μl of 50 mM Tris-HCl (pH8.0), 1 mM EDTA buffer. Then, 50 μl of lysis buffer (0.45% Tween 20, 0.45% Triton X-100, and 24 ug Proteinase K) was added, and incubated at 56° C. for 1 hour. The sample was boiled for 10 minutes, briefly chilled on ice for one minute, then centrifuged at 12,000× g for 10 minutes to pellet the cell debris. The DNA was ethanol precipitated from the supernatant and resuspended in 100 μl of 50 mM Tris-HCl (pH8.0), 1 mM EDTA buffer. Five μl of DNA was used directly for the PCR reaction supplemented with an additional 5 μl of sterile ddH₂O.

10 μl of each template was amplified in a 50 μl standard PCR reaction using the CODEHOP algorithm 60 program (9 min. 94° C. (No “Hot Start” (standard Taq)) then 50 cycles of 94° C. (30 sec.), 55° C. (30 sec.), 72° C. (30 sec.), and a final extension at 72° C. for 5 minutes. 5 μl of each reaction was analyzed on a 1% agarose gel.

The Quinolone-Resistance-Determining-Regions (QRDRs) of the topoisomerase IV gene parC was amplified by PCR from prokaryotic DNA by using the following mixture.

| | | Final |
|---|---|---|
| Components | Volume (μl) | Concentration |
| Genomic DNA | 10 | — |
| 10× PCR Buffer | 5 | 1X |
| 10 mM dNTP mixture | 1 | 0.2 mM each |
| Primers (25 pmol/μl) | 2 | 100 pmoles each |
| 50 mM MgCl₂ | 1.5 | 1.5 mM |
| Taq DNA polymerase (Roche) | 0.5 | 2.5 units |
| Sterile ddH₂O | 30 | — |

Degenerate QRDR primers designed to amplify the parC genes from various bacterial species are shown below.

| | |
|---|---|
| parC Deg.For | |
| 5′-CGCGATGGCCTGAAACCNGTNCARMG-3′ | [SEQ ID NO:5] |
| parC Deg.Rev | |
| 5′-AGGCGCGCTTCGGTATANCKCATNGCNGC-3′ | [SEQ ID NO:6] |

Table 1 illustrates which QRDR regions were amplified by the conditions described in Example 3. The following QRDR regions of the topoisomerase IV gene parC was amplified by PCR with an annealing temperature of 55° C., 50 reaction cycles, and Taq DNA polymerase without a “Hot Start”.

TABLE 1

| | Example 2 | | Example 3 |
|---|---|---|---|
| | Results | | Results |
| Species | gyrA | gyrB | parC |
| Acinetobacter baumanii | Yes | Yes | Yes |
| Acinetobacter calcoaceticus | Yes | Yes | |
| Cirrobacter freundii | Yes | Yes | |
| Corynebacterium xerosis | Yes | | |
| Enterobacter aerogenes | Yes | | |
| Enterobacter cloacae | Yes | Yes | |
| Escherichia coli | Yes | Yes | Yes |
| Kleibsiella oxytoca | Yes | Yes | Yes |
| Kleibsiella pneumoniae | Yes | | |
| Morganella morganii | | Yes | Yes |
| Micrococcus luteus | Yes | | |
| Proteus mirabilis | Yes | Yes | Yes |
| Providenica spp. | Yes | | Yes |
| Psuedomonas aeruginosa | Yes | | Yes |
| Serratia marcessens | | Yes | |
| Stenotrophomonas maltophilia | Yes | Yes | Yes |

Each reference cited herein is hereby incorporated by reference in its entirety. Moreover, each patent application to which this application claims priority is hereby incorporated by reference in its entirety.

What is claimed is:

1. A method for amplifying a polynucleotide sequence of a QRDR comprising the steps of:
(a) providing a composition comprising a degenerate forward primer of the invention and degenerate reverse primer of the invention, and a sample suspected to have a polynucleotide comprising a QRDR; and
(b) amplifying a QRDR.

2. The method of claim 1 wherein a primer is labeled.

3. The method of claim 1 wherein a primer is between 10 and 30 nucleotides in length.

4. The method of claim 1 whereby the amplifying step comprises PCR.

5. The method of claim 1 whereby the amplifying step (b) comprises between about 40 to 50 reaction cycles.

6. A method for identifying a polymorphic polynucleotide sequence of a QRDR comprising the steps of:
(a) providing a composition comprising a degenerate forward primer of the invention and/or and a degenerate reverse primer of the invention, and a sample suspected to have a polynucleotide comprising a QRDR region;
(b) amplifying a QRDR to obtain an amplified product;
(c) sequencing said amplified product to obtain a first polynucleotide sequence; and
(d) comparing said first polynucleotide sequence with a second polynucleotide sequence of an amplified product made using said degenerate forward primer primer of the invention and said degenerate reverse primer primer of the invention to identify sequence differences between said first polynucleotide sequence and said second polynucleotide sequence.

7. The method of claim 6 wherein a primer is labeled.

8. The method of claim 6 wherein a primer is between 10 and 30 nucleotides in length.

9. The method of claim 6 whereby the an amplifying step comprises PCR.

10. The method of claim 6 whereby the amplifying step (b) comprises between about 40 to 50 reaction cycles.

11. The method of claim 1 or 6 wherein said QRDR is amplified from a member of the genus selected from the group consisting of Psuedomonas, Enterococcus, Staphylococcus, Escherichia, Acinetobacter, Citrobacter, Corynebacterium, Enterobacter, Klebsiella, Morganella, Micrococcus, Proteus, Providenica, Serratia, and Stenotrophomonas.

12. The method of claim 1 or 6 wherein said QRDR is amplified from a member of the species selected from the group consisting of Psuedomonas aeruginosa, Enterococcus faecalis, Staphylococcus aureus, Staphylococcus epidermidis, Escherichia coli, Acinetobacter baumanii, Acinetobacter calcoaceticus, Citrobacter freundii, Corynebacterium xerosis, Enterobacter aerogenes, Enterobacter cloacae, Kleibsiella pneumoniae, Kleibsiella oxytoca, Morganella morganii, Micrococcus luteus, Proteus mirabilis, Providenica spp., Serratia marcessens, and Stenotrophomonas maltophilia.

13. The method of claim 1 or 6 wherein said QRDR is from a gene selected from the group consisting of gyrA, gyrB, parC, and parE.

14. A polynucleotide selected from the group consisting of:
[SEQ ID NO:1]
5′-CCGGATGTGCGCGAYGGNYTNAA-3′;
[SEQ ID NO:2]
5′-GGTTATGCGGCGGAATGTTNGTNGCCATNCC-3′;
[SEQ ID NO:3]
5′-CGAACTGTTTCTGGTGGAAGGNGAYWSNGC-3′;
[SEQ ID NO:4]
5′-ATACAGCGGCGGCTGNGCDATRTANAC-3′;
[SEQ ID NO:5]
5′-CGCGATGGCCTGAAACCNGTNCARMG-3′;
[SEQ ID NO:6]
5′-AGGCGCGCTTCGGTATANCKCATNGCNGC-3′;
[SEQ ID NO:7]
5′-CAGTTTGAAGGNCARACNAA-3′; and
[SEQ ID NO:8]
5′-AATATGCGCGCCATCGSWRTCNGCRTC-3′.

15. A composition comprising the primer of claim 14.

16. A method for identifying a polynucleotide sequence of a QRDR comprising the steps of:
(a) providing a composition comprising a degenerate primer of the present invention suitable for use in hybridizations, which comprises a solid surface on which is immobilized at pre-defined regions thereon a plurality of defined oligonucleotide/polynucleotide sequences for hybridization; and
(b) identifying, sequencing, and characterizing genes which are implicated in disease, infection, or development and the use of such identified genes and the proteins encoded thereby in diagnosis, prognosis, therapy and drug discovery.
